(19) United States
(12) Patent Application Publication
(10) Pub. No.: US 2022/0071556 A1
(43) Pub. Date: Mar. 10, 2022

(54) System and Method for Providing Biofeedback Controls to Various Media Based Upon the Remote Monitoring of Life Signs

(71) Applicant: Miku, Inc., Woodbridge, NJ (US)

(72) Inventors: Eric G. White, Tinton Falls, NJ (US); David Robert Abrams, Jersey City, NJ (US)

(73) Assignee: Miku, Inc.

(21) Appl. No.: 17/013,206

(22) Filed: Sep. 4, 2020

(51) Int. Cl. A61B 5/00; A61B 5/08

(52) U.S. Cl. CPC A61B 5/486; A61B 5/0816; A61B 2560/0431; A61B 5/7415; A61B 5/7235; A61B 5/742

(57) ABSTRACT

A system and method for generating biofeedback using feedback signals created by at least one media device. A subject person is monitored to obtain biometric data. The biometric data is used to generate a biometric waveform. The biometric waveform is used to generate control signals. The control signals are used to regulate audio signals, lighting, audiovisual imagery and/or auxiliary devices so that these feedbacks are altered to represent some waveform characteristic embodied by the biometric waveform. The subject person will hear and/or see the feedback and will better be able to control their body and mind.

### BACKGROUND OF THE INVENTION

### 1. Field of the Invention

In general, the present invention relates to systems and methods that monitor one or more life signs of a person and provide biofeedback data that is useful during meditation or to manage stress. More particularly, the present invention relates to system that use detected biofeedback data to control auxiliary media devices that can produce visual, audio and/or video stimuli.

### 2. Prior Art Description

In many forms of meditation, a person becomes more mindful of their body and mind, such as the movement of their chest and abdomen as they breathe, their heartbeat patterns, their thoughts, and any other body sensations. Using various meditative techniques, that person attempts to become more aware of how their body and mind respond to the way in which they are breathing. By controlling the way in which a person inhales and exhales, that person can cause a desired effect on the body and mind. For some people, meditation may be assisted by using a biofeedback device that can detect and respond to the change in a person's body. Biofeedback devices can monitor respiration rate, heart rate and other physical changes, and provide real-time feedback. In the same way a teacher may help identify and correct a student's knowledge of the subject matter that the student may not be aware they are lacking in, this biofeedback device may help identify and correct for habits in a user's meditation practices that they may not be aware could be improved upon.

There are many devices that are designed to monitor life signs of a person. In the prior art, respiration rate and heartrate can be detected either actively or remotely. Active detection systems place sensors on the body. For example, respiration rate can be actively detected by placing a sensor in the mouth that detects air as it is inhaled and exhaled. Such prior art is exemplified by U.S. Patent Application Publication No. 2015/0011906 to Wallach. Alternatively, a sensor can be placed around the chest that detects the expansion and contraction of the chest during respiration. Such prior art devices are exemplified by U.S. Pat. No. 9,699,528 to Dixit. Such active systems can obtain detailed breathing waveforms.

It will be understood that if a person is attempting to meditate, having a sensor in the mouth or wrapped around the chest can be a significant distraction. As a result, many people prefer to use remote sensors that do not restrict the movements of a person or detract from the comfort of that person. In the prior art, it has been possible to detect respiration rate and/or heart rate remotely using radar, lidar, cameras, piezoelectric sensors and the like. Radar systems are exemplified by Chinese Patent Disclosure No. CN104133199A and Chinese Patent Disclosure No. CN103110422A. Radar systems are exemplified by U.S. Patent Application Publication No. 2019/0139389 to White et al. However, such systems are used to detect the existence of life signs. Such devices are used in hospitals or as baby monitors, wherein an alarm is sounded if a life sign indicates a health emergency or values fall outside of the normal range.

In the prior art, there are systems that detect respiration rate or heartrate and use this information to adjust an external system. For example, CPAP breathing machines detect respiration and automatically synchronizes its operation to the respiration rate. Systems also exist that select and synchronize music to breathing rate or heartrate, such systems are exemplified by U.S. Pat. No. 10,327,073 to McElhone. However, many of these prior art systems contain circuitry and software that add latency to the detected signals as they are processed. The latency in signal analytics makes such prior art systems inadequate for biofeedback applications. Biometric signals used by a biofeedback system must be time synchronized and amplitude synchronized to the movements of the body. If the biometric signals were delayed or not time aligned with the user's body, then the feedback would not have a meditative, relaxing or entertaining benefit. It is also important the amplitudes be accurate in real-time so the biofeedback can vary in intensity. Measuring the respiration rate of the heart rate alone does not give adequate information to accomplish the needs of biofeedback for meditation, relaxation or entertainment.

A need therefore exists for a system and method that can integrate data contained within a breathing waveform or a heartbeat waveform into a real-time biofeedback system that is appropriate for meditation, relaxation or entertainment. A need also exists for such a system that is responsive, accurate, and portable. In this manner, a person can meditate with detailed feedback at various locations. These needs are met by the present invention as described and claimed below.

### SUMMARY OF THE INVENTION

The present invention is a system and method for generating biofeedback using feedback signals created by at least one media device. A subject person is scanned to obtain biometric data. The biometric data is used to generate a waveform, wherein the waveform has waveform characteristics other than frequency that are indicative of the body's physical position as a person breathes and/or as their heart beats. The waveform is used to generate low-latency control signals.

The control signals are used to regulate audio signals, lighting, and/or audiovisual imagery so that these feedback signals can be altered to represent the physical waveform characteristics of the user's body at each moment in time. The subject person will hear, feel and/or see the feedback signals and that align with their real-time breathing and/or heartrate.

### BRIEF DESCRIPTION OF THE DRAWINGS

For a better understanding of the present invention, reference is made to the following description of exemplary embodiments thereof, considered in conjunction with the accompanying drawings, in which:

FIG. 1 shows an overview schematic of an exemplary embodiment of the present invention biofeedback control system;

FIG. 2 shows graphs that include a biometric waveform synchronized in time with various audio signals that are controlled by the biometric waveform;

FIG. 3 shows graphs that include a biometric waveform synchronized in time with various light signals that are controlled by the biometric waveform;

FIG. 4 shows graphs that include a biometric waveform synchronized in time with audiovisual imagery that is controlled by the biometric waveform;

FIG. 5 is a block diagram that shows operational details of the biofeedback control system;

FIG. 6 shows an overview schematic of a first alternate embodiment of the present invention biofeedback control system;

FIG. 7 shows an overview schematic of a second alternate embodiment of the present invention biofeedback control system; and

FIG. 8 shows an overview schematic of a third alternate embodiment of the present invention biofeedback control system.

### DETAILED DESCRIPTION OF THE DRAWINGS

The present invention biofeedback control system and method can be can embodied in many ways. A few exemplary embodiments of the monitoring system have been selected for the purposes of description and illustration that show the present invention being used by an individual who wants to meditate. The illustrated embodiments are merely exemplary and should not be considered limitations when interpreting the scope of the appended claims. Furthermore, it should be understood that although intended for meditation, the present invention can be utilized for other purposes, such as entertainment, stress relief, anxiety treatment and to treat a variety of mental health conditions.

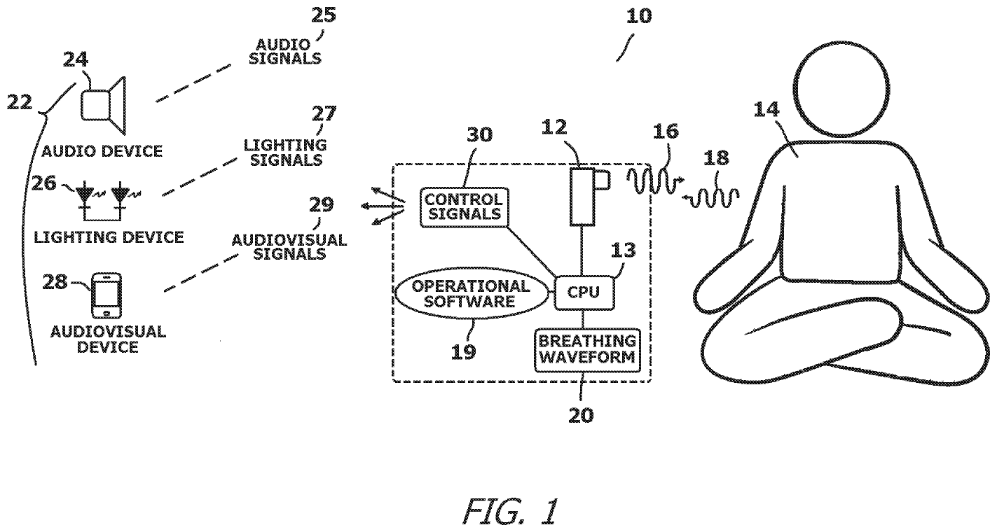

Referring to FIG. 1, an overview of an exemplary biofeedback control system 10 is shown. The biofeedback control system, 10 requires that the breathing waveform and/or the heartbeat waveform of a person be obtained. This can be accomplished in different ways, such as with the use of biomedical sensors. However, in the preferred embodiment, the biofeedback control system 10 includes a monitoring unit 12. The monitoring unit 12 is placed in a room and is directed toward a subject person 14. In the preferred embodiment, the monitoring unit 12 does not contact the subject person 14. Rather, the monitoring unit 12 actively emits scanning signals 16, such as lidar signals or radar signals. The scanning signals 16 are not visible or audible to the subject person 14. In this manner, the scanning signals 16 do not serve as a distraction to the subject person 14 attempting to meditate. Furthermore, the scanning signals 16 are low-energy signals that are harmless to the subject person 14 and any other sensitive electronic equipment, such as a pacemaker, that may be present.

The monitoring unit 12 emits the scanning signals 16 and receives back reflected signals 18 that reflect from the subject person 14. The monitoring unit 12 has a central processing unit 13 that runs operational software 19. The monitoring unit 12 detects the reflected signals 18 and uses circuitry and processing software to specifically extract biometric data from the reflected signals 18 that are associated with the breathing and/or heartbeat of the subject person 14. The extracted biometric data is then used to generate a biometric waveform 20, that can be either a heartbeat waveform or the illustrated breathing waveform. The methodology used to generate biometric waveforms 20 is disclosed in U.S. Patent Application Publication No. 2019/0139389 to White et al, the disclosure of which is herein incorporated by reference.

As will be explained, the biometric waveform 20 identified by the monitoring unit 12 is used to produce control signals 30 that control the output of various media devices 22. The control signals 30 are generated in real time with a latency that is unperceivable by the user. In this manner, the control of the media devices 22 seems synchronized with user's biometrics. The media devices 22 include audio devices 24, lighting devices 26 and auxiliary devices 28. The audio devices 24 are devices, such as digital music players and virtual assistant AIs, that broadcast audio signals 25 in the form of music, tones, spoken words, or any other audio. The lighting devices 26 are devices, such as lighting fixtures and LED arrays, that produce light signals 27 in the form of polychromatic, monochromatic, or any other kind of light. The auxiliary devices 28 are electronic devices, such as televisions smartphones, scent infusers or the like that produce other perceptible signals 29. The media devices 22 can be integrated into the monitoring unit 12. If mot, the media devices 22 receive control signals 30 from the monitoring unit 12. This can be done using either cables or by establishing a wireless data link, such as a Bluetooth® data link.

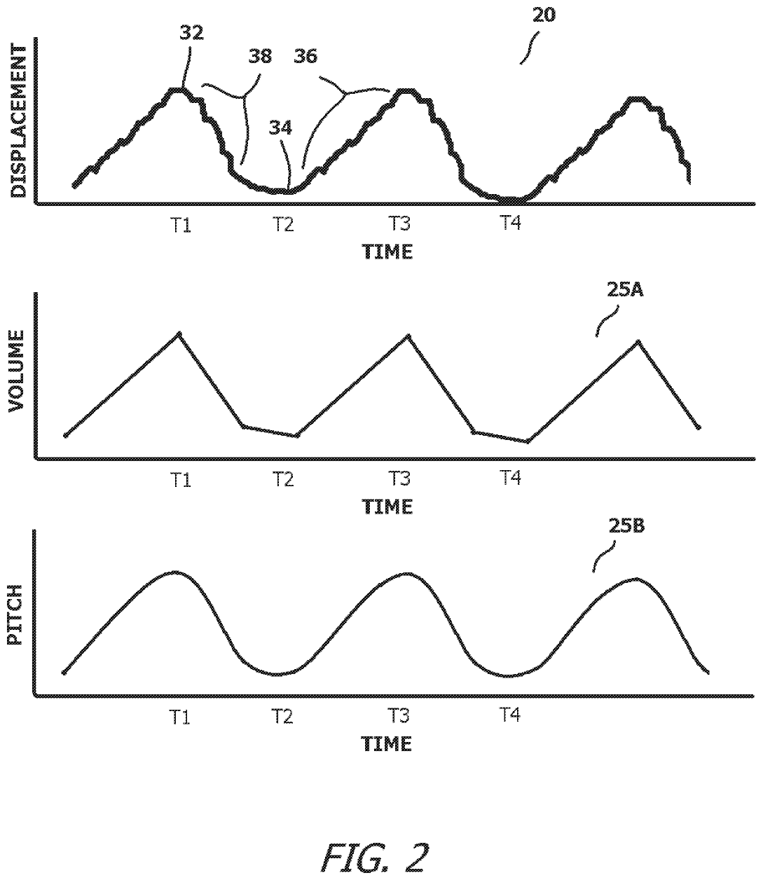

Referring to FIG. 2 in conjunction with FIG. 1, the biometric waveform 20 being shown is a breathing waveform. It will be understood that a heartbeat waveform can also be used. However, a breathing waveform is illustrated since a person has more conscious control over breathing than they do over their heartbeat. As such, a breathing waveform is more sensitive to biofeedback. From the figures, it can be seen that the exemplary biometric waveform 20 that is identified by the monitoring unit 12 has wave peaks 32 that alternate with wave troughs 34. In the exemplary biometric waveform 20, the wave peaks 32 occur at time positions T1 and T3. The wave troughs 34 occur at time positions T2 and T4. The wave peaks 32 and the wave troughs 34 are connected by ascending wave sections 36 and descending wave sections 38. The ascending wave sections 36 occur between time positions T2 and T3. The descending wave sections 38 occur between time positions T1 and T2. The periodicity of the wave peaks 32 and the wave troughs 34 is determined by the rate of breathing of the subject person 14 at any given moment in time. Typically, the faster the subject person 14 is breathing, the shorter the rise and fall times of the biometric waveform 20. Furthermore, the biometric waveform 20 has an amplitude that corresponds to the vertical distance between the wave peaks 32 and the wave troughs 34. The amplitude of the biometric waveform 20 corresponds to the degree of chest displacement when breathing. Deep breaths have larger amplitudes than do shallow breaths.

The biometric waveform 20 is used to generate corresponding control signals 30. The control signals 30 are generated and executed in a fraction of a second, so as to provide no perceivable latency to the user. As mentioned, the control signals 30 can be used to control various media devices 22 that produce audio signals 25, light signals 27 and/or perceptible signals 29. In FIG. 2, a first audio signal 25A is shown that is synchronized in time with the biometric waveform 20. The first audio signal 25A can be a tone, music, or any other audible sound. The first audio signal 25A is produced by an audio device 24 that is controlled by the control signal 30 generated by the monitoring unit 12. As can be seen, the first audio signal 25A can be controlled in volume by the control signal 30. The volume can be caused to increase in intensity during times (T2-to-T3) when the biometric waveform 20 is ascending. Likewise, the volume can be caused to decrease in intensity during times (T1-to-T2) when the biometric waveform 20 is descending. The volume of the first audio signal 25A, therefore, peaks in time with the peaks of the biometric waveform 20. The audio signal, therefore, varies with features of the biometric waveform other than just frequency. The features may include inflection points, rise time, fall time, acceleration rate, deceleration rate, pauses in breathing and the like. As such, the subject person 14 meditating will hear the first audio signal 25A as it increases and decreases in volume while the subject person 14 breaths. This provides audio feedback that can help the subject person 14 regulate his/her breathing.

Also shown in FIG. 2 is a second audio signal 25B that is synchronized in time with the biometric waveform 20. The second audio signal 25B can be a tone or a series of tones, that change in frequency as a function of the biometric waveform 20. The second audio signal 25B is produced by an audio device 24 that is controlled by the control signal 30 generated by the monitoring unit 12. As can be seen, the second audio signal 25B can be controlled in pitch by the control signal 30. The volume can be caused to increase in frequency during times (T2-to-T3) when the biometric waveform 20 is ascending. Likewise, the frequency can decrease during times (T1-to-T2) when the biometric waveform 20 is descending. The frequency of the second audio signal 25B, therefore, peaks in time with the biometric waveform 20. The audio signal, therefore, varies with features of the biometric waveform other than just frequency. As such, the subject person 14 meditating will hear an audio signal that changes in frequency as the subject person 14 breathes. This provides audio feedback that can help the subject person 14 regulate his/her breathing.

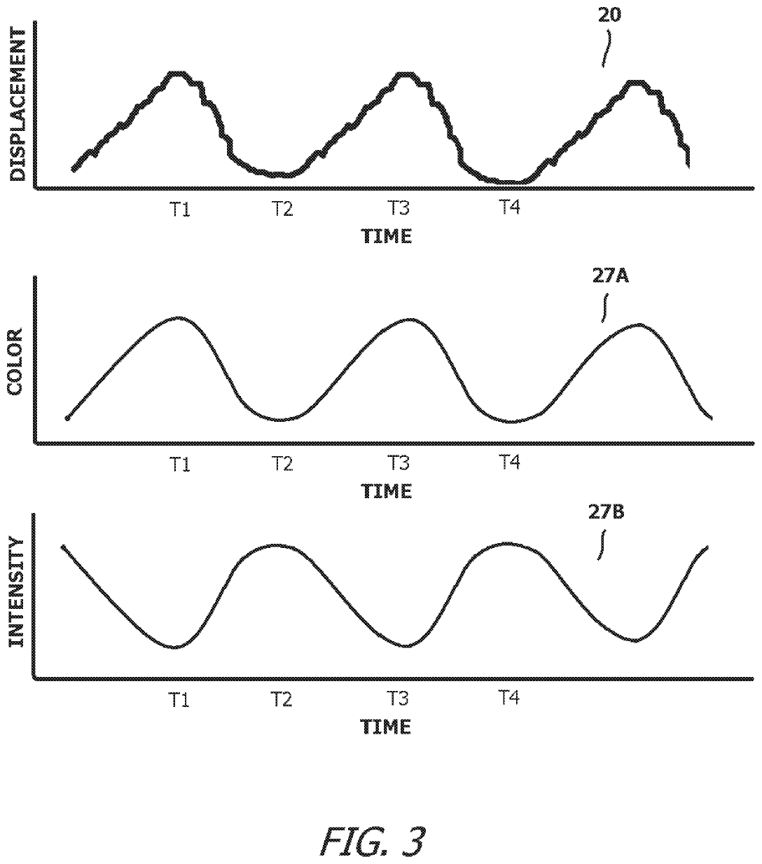

Referring to FIG. 3 in conjunction with FIG. 1, it will be understood that the control signals 30 generated from the biometric waveform 20 can be used to control various lighting devices 26 that produce light signals 27, such as an adjustable light fixture or an array of LEDs. In FIG. 3, a first light signal 27A is shown that is synchronized in time with the biometric waveform 20. As can be seen, the first light signal 27A can be controlled in frequency (i.e. color) by the control signal 30. By way of example, the color can transition from green toward red during times (T2-to-T3) when the biometric waveform 20 is ascending. Likewise, the color can change in frequency to transition back toward green during times (T1-to-T2) when the breathing waveform 20 is descending. The colors of the first light signal 27A therefore change in time with the biometric waveform 20. As such, the subject person 14 meditating will see colors that change as the subject person 14 breathes. This provides visual feedback that can help the subject person 14 regulate his/her breathing.

FIG. 3 also illustrates a second light signal 27B that is shown that is synchronized in time with the biometric waveform 20. The second light signal 27B can be a polychromatic or monochromatic light that changes in intensity as a function of the biometric waveform 20. The second light signal 27B is produced by the lighting device 26 that is controlled by the control signal 30 generated by the monitoring unit 12. The intensity of the second light signal 27B can be caused to increase during times (T2-to-T3) when the biometric waveform 20 is ascending. Likewise, the intensity can decrease during times (T1-to-T2) when the biometric waveform 20 is descending. The intensity of the second light signal 27B therefore peaks in time with the biometric waveform 20. As such, the subject person 14 meditating will see the changes in intensity as the subject person 14 breathes. This provides visual feedback that can help the subject person 14 regulate his/her breathing.

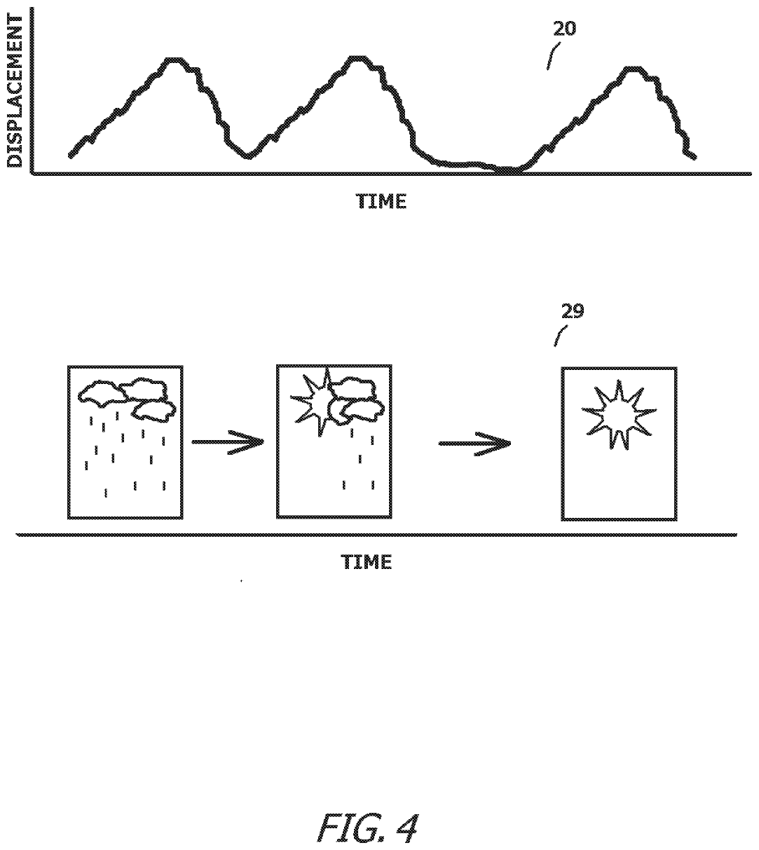

Referring to FIG. 4 in conjunction with FIG. 1, it will be understood that the control signal 30 generated from the biometric waveform 20 can be used to control various auxiliary devices 28 such as a smartphone, that produce audio visual signals or other perceptible signals 29. In FIG. 4, the perceptible signal 29 is imagery and sounds that are synchronized in time with the biometric waveform 20. As can be seen, the audiovisual signal 29 can be a video of a scene that changes in time. As illustrated, the video is a skyscape that can change between rainy and sunny. By way of example, the video imagery can transition from rainy toward sunny should the frequency of the biometric waveform 20 slow. Conversely, the video imagery can transition back toward rainy should the frequency of the biometric waveform 20 increase. As such, the subject person 14 meditating will see imagery that changes as that person's respiration rate changes. This provides audiovisual feedback that can help the subject person 14 regulate his/her breathing.

From the above, it will be understood that audio signals 25, light signals 27 and/or perceptible signals 29 can be varied in a variety of ways by the control signal 30. The control signal 30 can control the audio signals 25, light signals 27 and/or perceptible signals 29 to be time synchronized and amplitude synchronized with the biometric waveform 20. Alternatively, the audio signals 25, light signals 27 and/or perceptible signals 29 can be altered as a function of the frequency (i.e. respiration rate) of the biometric waveform 20 and/or by a rate of change in amplitude (i.e. alterations in deepness of breaths) of the biometric waveform 20.

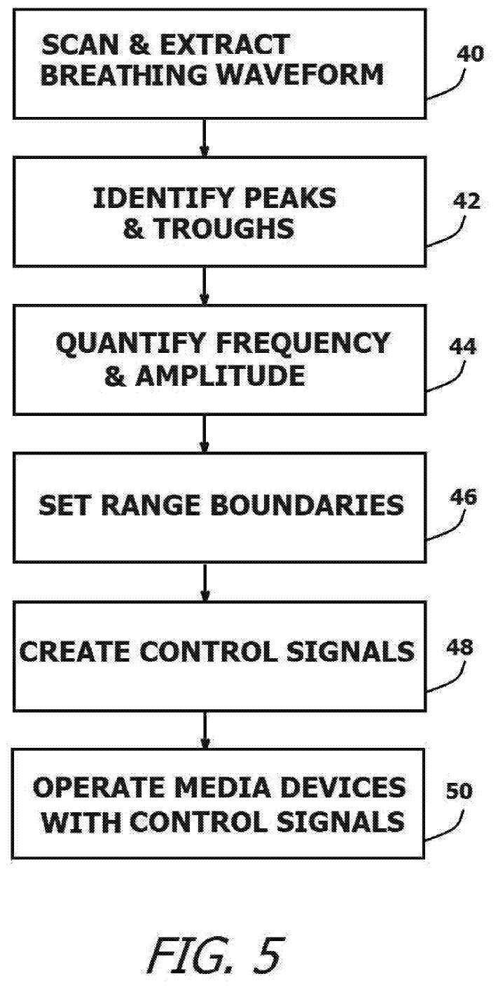

Referring to FIG. 5 in conjunction with FIG. 1, it will now be understood that the monitoring unit 12 extracts the biometric waveform 20 from the reflected signals 18 that reflect from the subject person 14. See Block 40. Using the operational software 19, the monitoring unit 12 detects the wave peaks 32 and wave troughs 34 embodied by the biometric waveform 20. See Block 42. With the wave peaks 32 and wave troughs 34 identified, changes in frequency and amplitude over time can be determined. See Block 44. Changes in frequency coincide with changes in respiration rate. Changes in amplitude coincide with changes in the depth of breathing.

Optionally, range boundaries for the frequency and amplitude of the biometric waveform 20 can be calculated or set. Alternatively, the operational software 19 can calculate a person's average respiration rate and average breathing amplitude through direct measurements over time. These average values can then be used to determine appropriate high/low ranges for the audio signals 25, light signals 27 and/or perceptible signals 29.

Using the high/low ranges, the monitoring unit 12 generates control signals 30 that are a function of the biometric waveform 20. See Block 48. The control signals 30 are sent to the various media devices 22 that are used by the biofeedback control system 10 in order to operate those media devices 22. See Block 50. The media devices 22 create the audio signals 25, light signals 27 and/or perceptible signals 29 bounded within the selected high/low ranges. In this manner, what might be considered a red line for one user may not be for another user. Rather, the biofeedback control system 10 adjusts to the life signs of the subject person 14.

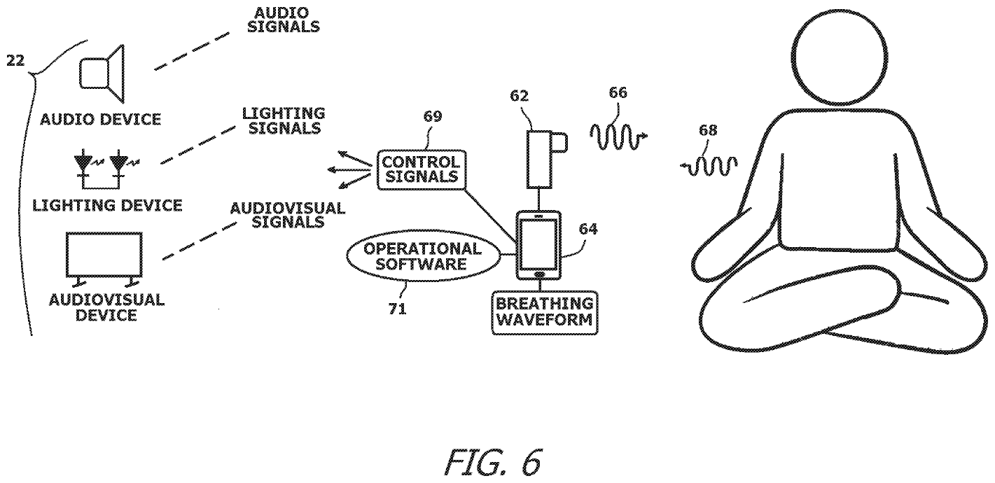

In the embodiment of the biofeedback control system 10 described above, the monitoring unit 12 contains the central processing unit 13 that runs the operational software 19. It is the operational software 19 that identifies the biometric waveform 20 and creates the corresponding control signals 30. Referring to FIG. 6, an alternate embodiment of a biofeedback control system 60 is shown. In this embodiment, a monitoring unit 62 is linked to a computing device 64, such as a tablet computer or a smartphone. The monitoring unit 62 contains the circuitry and components needed to broadcast scanning signals 66 and detect reflected signals 68. However, the monitoring unit 62 does not contains a processing unit. Rather, the monitoring unit 62 is controlled by the computing device 64. The computing device 64 receives the data for the reflected signals 68 from the monitoring unit 62. The computing device 64 runs operational software 71 that generates control signals 69 and forwards the control signals 69 to any media devices 22 in use.

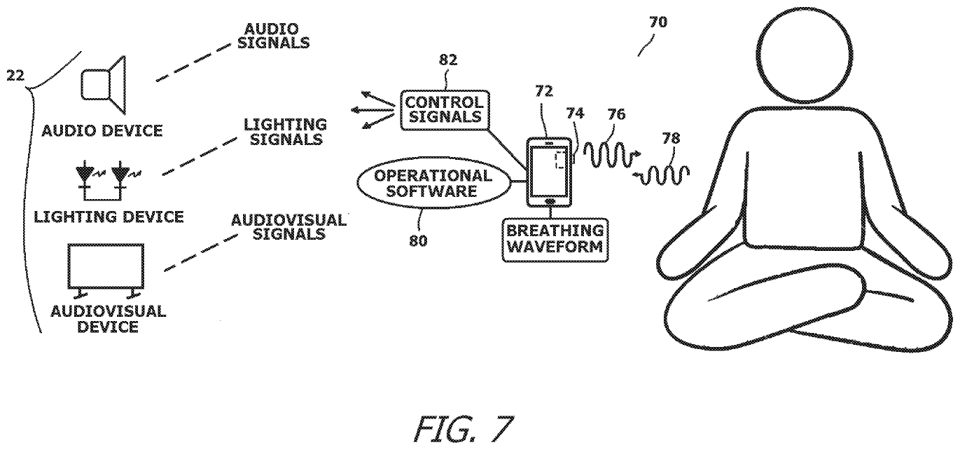

Modern smartphones use depth mapping in order to perform autofocus features. Some smartphones are being equipped with lidar scanners by the manufacturers. Referring to FIG. 7, an alternate embodiment of a biofeedback control system 70 is shown. In this embodiment, no separate monitoring unit is used. Rather a smartphone 72 is used that contains an internal lidar system 74. The lidar system 74 enables the smartphone 72 to generate scanning signals 76 and receive back reflected signals 78. The smartphone 72 processes the reflected signals 78. The smartphone 72 runs operational software 80 that generates the control signals 82 and forwards the control signals 82 to any media devices 22 in use.

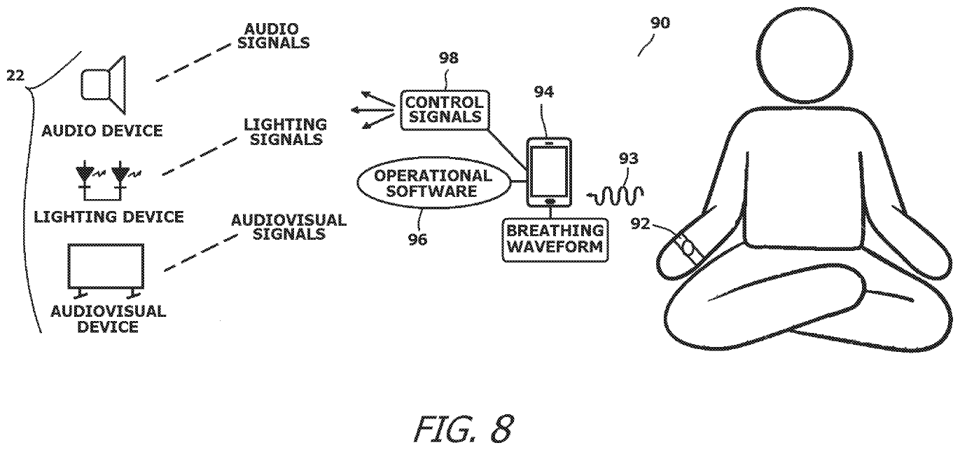

In all prior exemplary embodiments, the biometric waveform was obtained remotely. This need not be a limitation. The biometric waveform can be obtained in active manners, using a variety of sensors. There are many fitness monitors sold in commerce that are capable of detecting heartbeat waveforms and/or biometric waveforms. Such devices typically communicate with software being run on a smartphone, via a Bluetooth® connection. Referring to FIG. 8, an alternate embodiment of a biofeedback control system 90 is shown. In this embodiment, a biomonitor 92 is worn by the person trying to meditate. The biomonitor 92 can detect biometric data in the form of a breathing waveform and/or a heartbeat waveform. Biometric data 93 is then transmitted to a smartphone 94. The biometric data 93 can be transferred using a cable or using a wireless transmission. The smartphone 94 processes the biometric data 93. The smartphone 94 runs operational software 96 that generates the control signals 98 and forwards the control signals 98 to any media devices 22 in use.

It will be understood that the embodiments of the present invention that are illustrated and described are merely exemplary and that a person skilled in the art can make many variations to those embodiments. All such embodiments are intended to be included within the scope of the present invention as defined by the appended claims.

What is claimed is:

1. A method of generating biofeedback using feedback signals created by at least one media device, said method comprising the steps of:
monitoring a subject person to obtain a biometric waveform, wherein said biometric waveform has waveform characteristics;
utilizing said biometric waveform to generate control signals; and
controlling said at least one media device with said control signals, wherein said at least one media device broadcasts said feedback signals as directed by said control signals.

2. The method according to claim 1, wherein said at least one media device is selected from a group consisting of audio devices, lighting devices and audiovisual devices.

3. The method according to claim 1, wherein said at least one media device is an audio device that broadcasts an audio signal that is selectively altered by said control signals to correspond with at least one of said waveform characteristics of said biometric waveform.

4. The method according to claim 1, wherein said at least one media device is an audio device that broadcasts an audio signal with a pitch frequency that is selectively altered by said control signals to correspond with at least one of said waveform characteristics of said biometric waveform.

5. The method according to claim 1, wherein said at least one media device is an audio device that broadcasts an audio signal that is selectively altered in volume by said control signals to correspond with at least one of said wave characteristics of said biometric waveform.

6. The method according to claim 1, wherein said at least one media device is a lighting device that emits light that is selectively altered by said control signals to correspond with at least one of said waveform characteristics of said biometric waveform.

7. The method according to claim 1, wherein said at least one media device is a lighting device that broadcasts light with a color profile that is selectively altered by said control signals to correspond with at least one of said waveform characteristics of said biometric waveform.

8. The method according to claim 1, wherein said at least one media device is a lighting device that broadcasts light with a light intensity that is selectively altered by said control signals to correspond with at least one of said wave characteristics of said biometric waveform.

9. The method according to claim 1, wherein said at least one media device is an audiovisual device that produces imagery that is selectively altered by said control signals to correspond with at least one of said waveform characteristics of said biometric waveform.

10. The method according to claim 1, wherein said wave characteristics of said waveform are selected from a group consisting of wave peaks, wave troughs, wave frequency, wave amplitude and changes thereto for a given period of time.

11. A method of generating biofeedback using feedback signals, said method comprising the steps of:
providing a media device that produces said feedback signals, wherein said feedback signals are selected from a group consisting of audio signals, light signals and audiovisual signals;
providing a biomonitoring device that can detect biometric data from a person, wherein said biometric data is selected from a group consisting of breathing waveforms and heartbeat waveforms, and wherein said biometric data contains waveform characteristics other than frequency;
utilizing said waveform characteristics to generate control signals;
controlling said media device with said control signals, wherein said media device broadcasts and alters said feedback signals as directed by said control signals.

12. The method according to claim 11, wherein said media device broadcasts an audio signal that is selectively altered by said control signals to correspond with at least one of said waveform characteristics.

13. The method according to claim 11, wherein said media device broadcasts an audio signal with a pitch frequency that is selectively altered by said control signals to correspond with at least one of said waveform characteristics.

14. The method according to claim 11, wherein said media device broadcasts an audio signal that is selectively altered in volume by said control signals to correspond with at least one of said wave characteristics.

15. The method according to claim 11, wherein said media device emits light that is selectively altered by said control signals to correspond with at least one of said waveform characteristics.

16. The method according to claim 11, wherein said media device broadcasts light with a color profile that is selectively altered by said control signals to correspond with at least one of said waveform characteristics.

17. The method according to claim 11, wherein said media device broadcasts light with a light intensity that is selectively altered by said control signals to correspond with at least one of said wave characteristics.

18. A method of generating biofeedback using feedback signals, said method comprising the steps of:
providing a portable electronic device capable of broadcasting said feedback signals, wherein said feedback signals are selected from a group consisting of audio signals, light signals and audiovisual signals;
monitoring a subject person with a biomonitor to obtain biofeedback data;
using said biofeedback data to generate a waveform, wherein said waveform has waveform characteristics other than frequency;
utilizing said waveform to generate control signals;
controlling said feedback signals with said control signals to cause said feedback signals to reflect at least one of said waveform characteristics, wherein said media device broadcasts and alters said feedback signals as directed by said control signals.
